(19) United States
(12) Patent Application Publication
(10) Pub. No.: US 2008/0038342 A1
(43) Pub. Date: Feb. 14, 2008

(54) Stable Pharmaceutical Composition Comprising an Ace Inhibitor

(75) Inventors: Jeffrey Bergman, Herfordshire (GB); Pratibha S. Pilgaonkar, Mumbai (IN); Maharuk Tehmasp Rustomjee, Mumbai (IN); Amita P. Surana, Thane (IN); Rizwana Mulagath, Mumbai (IN); Atul A. Kelkar, Thane (IN)

Correspondence Address: WOLF GREENFIELD & SACKS, P.C., 600 ATLANTIC AVENUE, BOSTON, MA, 02210-2206, US

(73) Assignee: Niche Generics Limited, Hitchin (GB)

(21) Appl. No.: 10/590,816

(22) PCT Filed: Feb. 15, 2005
(86) PCT No.: PCT/GB05/50016

§ 371 Date: Jun. 28, 2007

(30) Foreign Application Priority Data
Feb. 27, 2004 (GB) 0404420.2

(51) Int. Cl. A61K 9/30; A61K 31/403; A61P 9/04; A61P 9/12; A61P 9/10; A61K 31/47

(52) U.S. Cl. 424/465; 514/307; 514/412; 514/777

(57) ABSTRACT

The present invention relates to a stable pharmaceutical composition comprising an ACE inhibitor or a pharmaceutically acceptable salt or derivative thereof. In particular, the invention relates to a pharmaceutical composition, which comprises an ACE inhibitor, or a pharmaceutically acceptable salt or a derivative thereof, and a C₁₆-C₂₈ glyceride. ACE inhibitors useful in the present invention are susceptible to heat and/or mechanical stress-induced degradation. Preferred ACE inhibitors are ramipril, trandolapril, quinapril and pharmaceutically acceptable salts and derivatives thereof. The composition of the present invention may be for use as a medicament for the treatment or prevention of a cardiovascular disease, a coronary heart disease, a cerebrovascular disease, a peripheral vascular disease, arrhythmia, hypertension, cardiac failure, cardiovascular death, myocardial infraction, stroke or angina. The present invention further relates to a method of preparing the pharmaceutical composition of the present invention. The present invention also relates to a method of providing a stable pharmaceutical composition comprising an ACE inhibitor, or a pharmaceutically acceptable salt or derivative thereof, by incorporating a C₁₆-C₂₈ glyceride into the composition. The present invention further relates to a use of C₁₆-C₂₈ glyceride to provide a stable pharmaceutical composition comprising an ACE inhibitor or a pharmaceutically acceptable salt or derivative thereof.

### TECHNICAL FIELD

The present invention relates to a stable pharmaceutical composition comprising an ACE inhibitor or a pharmaceutically acceptable salt or derivative thereof. In particular, the invention relates to a pharmaceutical composition, which comprises an ACE inhibitor, or a pharmaceutically acceptable salt or derivative thereof, and a C₁₆-C₂₈ glyceride. ACE inhibitors useful in the present invention are susceptible to heat and/or mechanical stress-induced degradation. Preferred ACE inhibitors are ramipril, trandolapril, quinapril and pharmaceutically acceptable salts and derivatives thereof. The composition of the present invention may be for use as a medicament for the treatment or prevention of a cardiovascular disease, a coronary heart disease, a cerebrovascular disease, a peripheral vascular disease, arrhythmia, hypertension, cardiac failure, cardiovascular death, myocardial infarction, stroke or angina.

The present invention further relates to a method of preparing the pharmaceutical composition of the present invention. The present invention also relates to a method of providing a stable pharmaceutical composition comprising an ACE inhibitor, or a pharmaceutically acceptable salt or derivative thereof, by incorporating a C₁₆-C₂₈ glyceride into the composition. The present invention further relates to a use of a C₁₆-C₂₈ glyceride to provide a stable pharmaceutical composition comprising an ACE inhibitor or a pharmaceutically acceptable salt or derivative thereof.

### BACKGROUND ART

ACE inhibitors, i.e. inhibitors of angiotensin converting enzymes, are drugs useful in the treatment of cardiovascular disorders, in particular hypertension and coronary heart disease. It has been widely observed that ACE inhibitors are susceptible to degradation between the time of manufacture and the time of desired usage, in particular due to cyclization, hydrolysis and oxidation. Typical degradation products are hydrolytic degradation products formed by hydrolysis of the ACE inhibitor and diketopiperazine degradation products formed by cyclization of the ACE inhibitor.

Ramipril, also called (2S,3aS,6aS)-1-[(2S)-[[(1S)-(ethoxycarbonyl)-3-phenylpropyl]amino]-1-oxopropyl]-octahydrocyclopenta[b]pyrrole-2-carboxylic acid, is an ACE inhibitor of formula 1. Trandolapril, also called (2S,3aR,7aS)-1-[(2S)-[[(1S-(ethoxycarbonyl)-3-phenylpropyl]amino]-1-oxopropyl]-octahydro-1H-indole-2-carboxylic acid, is an ACE inhibitor of formula 2. Quinapril, also called (3S)-2-[(2S)-[[(1S)-(ethoxycarbonyl)-3-phenylpropyl]amino]-1-oxopropyl]-1,2,3,4-tetra-hydroisoquinoline-3-carboxylic acid, is an ACE inhibitor of formula 3.

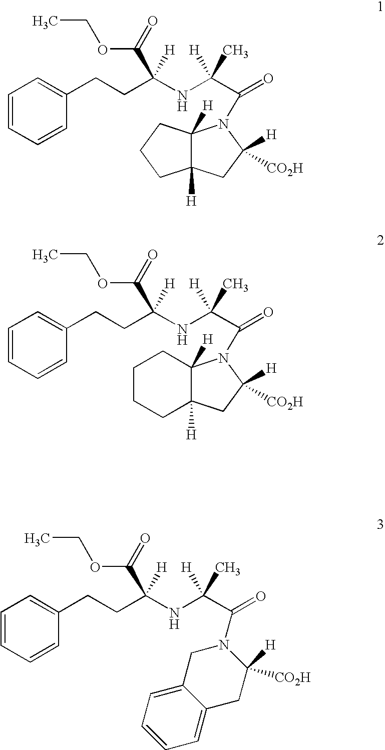

ACE inhibitors such as ramipril, trandolapril or quinapril, are used in the treatment or prevention of cardiovascular diseases, coronary heart diseases, peripheral vascular diseases, arrhythmias, hypertension, cardiac failure, cardiovascular death, myocardial infarction, stroke or angina.

Currently commercially available formulations of ramipril contain as inactive ingredients one or more of the following excipients: hydroxypropylcellulose, hydroxypropylmethylcellulose, mictocrystalline cellulose, pregelatinzed starch, maize starch, sodium steatyl fumatate, gelatin, anhydrous lactose, polyethylene glycol, polyoxyl hydrogenated castot oil, propyl gallate, sodium aluminium silicate, paraffin, and/or colouring agents (such as black, red and/or yellow ferric oxide E172, titanium dioxide E171, and/or indigo carmine E132).

Currently commercially available formulations of trandolapril contain as inactive ingredients one or more of the following excipients: corn starch, lactose, povidone, and/or sodium stearyl fumarate.

Currently commercially available formulations of quinapril contain as inactive ingredients one or more of the following excipients: magnesium carbonate, lactose, hydrous lactose, gelatin, povidone, crospovidone, magnesium stearate, candelilla wax, hydroxyptopylmethylcellulose, hydroxyptopylcellulose, polyethylene glycol, maize starch, talc, and/or colouring agents (such as red and/or yellow ferric oxide E172, titanium dioxide E171, and/or indigotine E132).

Many ACE inhibitors, including ramipril, trandolapril and quinapril, have an ester; (CO—O) and/or an amide (CO—N) bond. Such bonds are susceptible to hydrolysis leading to the formation of hydrolytic degradation products. Moreover, due to their molecular structure many ACE inhibitors, including ramipril, trandolapril and quinapril, are susceptible to cyclization to form diketopiperazine degradation products. Some known degradation products of ramipril are shown in FIG. 1, including hydrolytic degradation products E and F, and diketopiperazine degradation products D, K and L.

The degradation of ACE inhibitors has been found to occur both in solid and in liquid states. As the degradation of an ACE inhibitor in a pharmaceutical composition increases, the concentration of available, functional ACE inhibitor decreases. Thus the shelf-life of pharmaceutical compositions comprising the ACE inhibitor is limited due to this degradation. Accordingly, degradation should be avoided.

Various ways to mine the degradation of ACE inhibitors in pharmaceutical compositions have been advocated. For example, it has been suggested that alkali or alkaline-earth metal salts can stabilise ACE inhibitors and their salts and derivatives in pharmaceutical compositions.

WO 01/15724 and U.S. Pat. No. 6,555,551 disclose a method of stabilising pharmaceutical compositions comprising ACE inhibitors such as ramipril hydrochloride or quinapril hydrochloride. The method comprises the step of mixing an alcoholic dispersion of an ACE inhibitor with an aqueous solution or dispersion of a metal compound; the resulting mixture may be dried. Suitable metal compounds are alkali or alkaline-earth metal salts.

EP-0,280,999 and U.S. Pat. No. 4,743,450 teach that the cyclization, hydrolysis and discolouration of pharmaceutical compositions, comprising quinapril, enalapril, indolapril or structurally-related ACE inhibitors, are minimized by formulating the compositions with a metal-containing alkaline stabilizer. The metal-containing alkaline stabilizer is preferably an inorganic salt of an alkali or alkaline-earth metal, such as magnesium, calcium or sodium borate, silicate or carbonate.

WO 03/059388 discloses that the cyclization, hydrolysis and discolouration of pharmaceutical compositions, comprising ramipril, quinapril, trandolapril or structurally-related ACE inhibitors, are minimized by formulating the compositions with a basic compound and a filler. The basic compound is preferably an alkali or alkaline-earth metal carbonate, such as magnesium carbonate, sodium carbonate or sodium hydrogen carbonate. The filler is preferably an insoluble alkaline-earth metal hydrogen phosphate, such as calcium hydrogen phosphate.

WO 02/11709 discloses stable pharmaceutical compositions comprising ramipril and an effervescent system. The effervescent system comprises an alkali or alkaline-earth metal carbonate or bicarbonate, such as sodium, calcium or magnesium carbonate or bicarbonate, and at least one acid, such as citric acid, monosodium citrate, ascorbic acid, gluconic acid, lactic acid, malic acid or tartaric acid. The ratio of acid to (bi)carbonate is said to be between 0.6 and 1.3, and the ratio of ramipril to effervescent system is said to be between 0.004 and 0.013, for the pharmaceutical compositions to be stable.

WO 99/62560 and U.S. Pat. No. 6,417,196 disclose pharmaceutical compositions, comprising quinapril, enalapril, indolapril or structurally-related ACE inhibitors, which are stabilised by the presence of magnesium oxide, preferably in combination with a hydrolysis-minimizing agent. The presence of magnesium oxide is also said to lend itself to favourable processing conditions during the manufacture of the ACE inhibitor-containing compositions, especially processing by wet granulation.

It has also been suggested that certain acids can be used to stabilise ACE inhibitors in pharmaceutical compositions. EP-0,468,929, U.S. Pat. No. 6,300,361 and U.S. Pat. No. 6,300,362 disclose the use of hydrochloric acid donors as stabilizers in pharmaceutical compositions comprising ACE inhibitors such as quinapril, enalapril, spirapril, spiraptilate, ramipril, perindopril, indolapril, lisinopril, alacepril, trandolapril, benazepril, libenzapril, delaptil or cilazaptil. Suitable hydrochloric acid donors are amino acid hydrochlorides, such as glycine, glutamic acid, betaine, alanine, valine, lysine, arginine or aspartic acid hydrochloride, and Lewis acid chlorides, such as ferric, zinc or aluminium chloride.

Furthermore, it has been suggested that certain compounds such as lactose monohydrate can be used to stabilise ACE inhibitors such as ramipril in pharmaceutical compositions. WO 03/028707 discloses pharmaceutical compositions comprising ramipril and lactose monohydrate as diluent. The lactose monohydrate was found to stabilise the ramipril in the compositions. The compositions may further optionally comprise a lubricant, such as magnesium, zinc or calcium stearate.

Moreover, the use of protective coatings has been advocated to stabilise ACE inhibitors in pharmaceutical compositions. EP-0,317,878, U.S. Pat. No. 5,151,433 and U.S. Pat. No. 5,442,008 disclose pharmaceutical compositions comprising ACE inhibitors such as ramipril, enalapril, perindopril, indolapril, lisinopril, quinapril, alacepril or trandolapril, in which the ACE inhibitors are stabilised by a polymeric protective coating and/or by a buffer which maintains the pH of the compositions between 5.5 and 8.0.

WO 95/34283, EP-0,624,364 and U.S. Pat. No. 5,527,540 disclose pharmaceutical compositions comprising an alkali-sensitive active substance, such as captopril, ramipril, perindopril erbumine or enalapril, and an effervescent system, such as a carbonate component. To stabilise the active substance, it is embedded in at least one of the following compounds: an edible organic acid, a higher alcohol, a hydrocolloid, a long-chain polyvinylpyrrolidone, and is preferably coated with at least one of said compounds. The carbonate component is also preferably embedded in at least one edible organic acid and coated by the same or another acid.

Furthermore, in WO 03/059330 it has been suggested that mechanical stress-induced degradation of ACE inhibitors such as ramipril, spirapril, lisinopril, enalapril, quinapril, benazepril or structurally-related ACE inhibitors, can be avoided by coating a cote of diluents and other formulating agents with a layer of the ACE inhibitor. The core is compressed prior to coating with the ACE inhibitor, thereby avoiding the need to compress the ACE inhibitor and thus avoiding mechanical stress-induced degradation.

It has still further been suggested to stabilise ACE inhibitors by derivatisation. For example, WO 02/03970 discloses a transdermal therapeutic system comprising an adhesive matrix. The matrix comprises a derivative of an ACE inhibitor such as ramipril or trandolapril, which has been stabilised by derivatisation into a salt or diester.

Despite these efforts to stabilise ACE inhibitors, there remains a long-standing need for stable pharmaceutical compositions comprising an ACE inhibitor or a pharmaceutically acceptable salt or derivative thereof, and methods of preparing the same.

Surprisingly, it has now been found that the presence of a C₁₆-C₂₈ glyceride reduces or slows the degradation of ACE inhibitors such as ramipril, trandolapril, quinapril, or their salts or derivatives in pharmaceutical compositions. Astonishingly, until now C₁₆-C₂₈ glycerides such as glycerol dibehenate, a common pharmaceutical excipient, have not been used in pharmaceutical compositions comprising ramipril, trandolapril, quinapril, or their salts or derivatives either in the published prior art or in commercially available compositions.

### SUMMARY OF THE INVENTION

For the purposes of the present invention, a “C₁₆-C₂₈ glyceride” is a mono-, di- or tri-glyceride comprising one, two or three C₁₆-C₂₈ acyl moieties respectively. Preferably each C₁₆-C₂₈ acyl moiety is independently of the formula —CO—R, wherein R is a saturated or unsaturated hydrocarbon, which contains from 16 to 28 carbon atoms, and which is straight-chained or branched. Preferably R is a saturated hydrocarbon. Preferably R is a straight-chained hydrocarbon. The acyl moieties may be derived from naturally occurring or synthetic fatty acids. The terms “C₈-C₂₆ glyceride”, “C₂₀-C₂₄ glyceride” and “C₂₂ glyceride” are defined accordingly. Glycerol dibehenate comprises mainly C₂₂ diglyceride comprising two C₂₂ acyl moieties of the formula —CO—(CH)₂₀—CH₃.

A pharmaceutical composition comprising an ACE inhibitor, or a pharmaceutically acceptable salt or derivative thereof, is considered to be “stable”, if the ACE inhibitor, or its salt or derivative, in the pharmaceutical composition degrades less or more slowly than it does in known pharmaceutical compositions. The term “unstable” is defined accordingly.

An excipient is considered to be “compatible” with an ACE inhibitor, or a pharmaceutically acceptable salt or derivative thereof, if it does not promote the degradation of the ACE inhibitor, or its salt or derivative, i.e. if the ACE inhibitor, or its salt or derivative, does not degrades more or faster in the presence of the excipient compared to the degradation of the ACE inhibitor, or its salt or derivative, on its own. The terms “compatibility”, “incompatible” and “incompatibility” are defined accordingly.

An ACE inhibitor, or a pharmaceutically acceptable salt or derivative thereof, is considered to be “susceptible to heat and/or mechanical stress-induced degradation”, if it degrades more or faster when it is subjected to heat and/or mechanical stress such as, for example, due to pressure and heat exerted during compression of a powder blend into tablets, than it does when it is not subjected to heat and/or mechanical stress.

A drug, such as an ACE inhibitor or a pharmaceutically acceptable salt or derivative thereof, and an excipient, such as glycerol dibehenate, are considered to form a “mixture”, if the drug and the excipient are blended together. Thus, if a first excipient is solely used to coat a drug or a drug/second excipient blend, then the first excipient is not considered to form a mixture with the drug or the drug/second excipient blend. However, if an excipient is blended together with a drug and is also used to coat the drug/excipient blend, then the excipient is considered to form a mixture with the drug. A mixture or blend of a drug and an excipient is considered to form an “intimate mixture or blend”, if the mixture or blend is substantially uniform.

A first embodiment of the present invention provides a pharmaceutical composition comprising an ACE inhibitor, or a pharmaceutically acceptable salt or derivative thereof, and a C₁₆-C₂₈ glyceride. Preferably the pharmaceutical composition comprises 5-30% by weight C₁₆-C₂₈ glyceride, more preferably 5-20% by weight, even more preferably 10-15% by weight of the total composition.

Preferably the glyceride comprises one, two or three C₁₆-C₂₈ acyl moieties, wherein each C₁₆-C₂₈ acyl moiety is independently of the formula —CO—R, wherein R is a saturated or unsaturated hydrocarbon, which contains from 16 to 28 carbon atoms, and which is straight-chained or branched. Preferably R is a saturated hydrocarbon and/or R is a straight-chained hydrocarbon. Preferably the glyceride is a C₁₈-C₂₆ glyceride, more preferably a C₂₀-C₂₄ glyceride, even more preferably a C₂₂ glyceride. Preferably the glyceride comprises at least 50% diglyceride, more preferably at least 60% diglyceride, even mote preferably at least 70% diglyceride. In the most preferred embodiment of the present invention, the glyceride is glycerol dibehenate.

Preferably the ACE inhibitor, or the pharmaceutically acceptable salt or derivative thereof, is susceptible to heat and/or mechanical stress-induced degradation. More preferably the ACE inhibitor, or the pharmaceutically acceptable salt or derivative thereof, is ramipril, trandolapril quinapril, or a pharmaceutically acceptable salt or derivative thereof. In the most preferred embodiment of the present invention, the ACE inhibitor, or the pharmaceutically acceptable salt or derivative thereof, is ramipril or a pharmaceutically acceptable salt or derivative thereof.

Preferably the pharmaceutical composition comprises one or more further excipients, which are compatible with the ACE inhibitor or the pharmaceutically acceptable salt or derivative thereof.

The one or more further excipients may be selected from carbonates (such as calcium carbonate, sodium carbonate or magnesium carbonate), phosphates (such as anhydrous dibasic calcium phosphate, dibasic calcium phosphate dihydrate, tribasic calcium phosphate or sodium phosphate), sulfates (such as calcium sulfate), silicates (such as kaolin, talc, sodium aluminium silicate, magnesium aluminium silicate, magnesium silicate or magnesium trisilicate), carbohydrates (such as dextrates, dextrin, maltodextrin, dextrose, polydextrose, fructose, sucrose, sugar spheres, compressible sugar, confectioner's sugar, maltose, mannitol, lactose, anhydrous lactose, hydrous lactose, lactitol, maltitol sorbitol, sodium alginate, alginic acid or liquid glucose), starches (such as starch, pregelatinized starch, maize starch, corn starch or sodium starch glycolate), celluloses (such as carboxymethylcellulose calcium, carboxymethylcellulose sodium, cross-linked carboxymethylcellulose sodium, microcrystalline cellulose, silicified microcrystalline cellulose, powdered cellulose, cellulose acetate, cellulose acetate phthalate, methylcellulose, ethylcellulose, hydroxyethylcellulose, hydroxypropylcellulose, low-substituted hydroxypropylcellulose or hydroxypropylmethylcellulose), polyvinylpyrrolidones (such as povidone or crospovidone), fatty acids or fatty acid derivatives (such as hydrogenated vegetable oil, hydrogenated castor oil, polyoxyl hydrogenated castor oil, mineral oil, light mineral oil, cottonseed oil, a medium-chain triglyceride, glyceryl palmitostearate, calcium stearate, steatic acid, glyceryl monostearate, magnesium stearate, polyoxyethylene stearate, zinc stearate, sodium stearyl fumarate, candelilla wax or glycerol dibehenate), gums (such as tragacanth gum, guar gum or acacia), colouring agents (such as black, red or yellow ferric oxide, titanium dioxide or indigotine), magnesium oxide, sodium chloride, polymethacrylate, propyl gallate, colloidal silicon dioxide, polacrilin potassium, sodium lauryl sulfate, a poloxamer, polyethylene glycol, sodium benzoate, a carbomer, ceratonia, gelatin, paraffin, polyethylene oxide, zein, or a mixture thereof.

Preferably, the one or more further excipients are selected from carbonates (preferably magnesium carbonate), phosphates (preferably anhydrous dibasic calcium phosphate, dibasic calcium phosphate dihydrate or tribasic calcium phosphate), silicates (preferably kaolin, talc, sodium aluminium silicate, magnesium aluminium silicate, magnesium silicate or magnesium trisilicate), carbohydrates (preferably dextrates, maltodextrin, dextrose, polydextrose, fructose, sucrose, sugar spheres, compressible sugar, confectioner's sugar, maltose, mannitol, lactose, anhydrous lactose, hydrous lactose, lactitol, maltitol, sorbitol or sodium alginate), starches (preferably starch, pregelatinized starch, maize starch, corn starch or sodium starch glycolate), celluloses (preferably carboxymethylcellulose calcium, carboxymethylcellulose sodium, cross-linked carboxymethylcellulose sodium, microcrystalline cellulose, powdered cellulose, cellulose acetate, cellulose acetate phthalate, methylcellulose, ethylcellulose, hydroxyethylcellulose, hydroxypropylcellulose, low-substituted hydroxypropylcellulose or hydroxypropylmethylcellulose), polyvinylpyrrolidones (preferably povidone or crospovidone), fatty acids or fatty acid derivatives (preferably hydrogenated vegetable oil, hydrogenated castor oil, polyoxyl hydrogenated castor oil, glyceryl palmitostearate, calcium stearate, stearic acid, glyceryl monostearate, magnesium stearate, zinc stearate, sodium stearyl fumatate, candelilla wax or glycerol dibehenate), gums (preferably guar gum), colouring agents (preferably black, red or yellow ferric oxide, titanium dioxide or indigotine), sodium chloride, polymethacrylate, propyl gallate, colloidal silicon dioxide, sodium lauryl sulfate, a poloxamer, polyethylene glycol, sodium benzoate, a carbomer, ceratonia, gelatin, paraffin, polyethylene oxide, zein, or a mixture thereof.

More preferably, the one or more further excipients are selected from hydroxypropylmethylcellulose, pregelatimsed starch, microcrystalline cellulose, lactose, sodium starch glycolate, sodium stearyl fumarate, red ferric oxide and yellow ferric oxide.

Preferably the pharmaceutical composition comprises:
1-8% by weight ACE inhibitor, preferably 2-6% by weight;
5-20% by weight C₁₆-C₂₈ glyceride, preferably 10-15% by weight;
60-80% by weight lactose anhydrous, preferably 65-75% by weight;
5-20% by weight sodium starch glycolate, preferably 10-15% by weight;
0.5-4 by weight sodium stearyl fumarate, preferably 0.5-2% by weight;
0-0.4% by weight yellow ferric oxide; and
0-0.1% by weight red ferric oxide.

Preferably the pharmaceutical composition of the present invention is stable.

Preferably the pharmaceutical composition of the present invention is suitable for direct compression into tablets.

Preferably the ACE inhibitor, or the pharmaceutically acceptable salt or derivative thereof, and the C₁₆-C₂₈ glyceride forth a mixture, preferably an intimate mixture, in the pharmaceutical composition of the present invention. If one or more further excipients are present in the composition, preferably the ACE inhibitor, or the pharmaceutically acceptable salt or derivative thereof, the C₁₆-C₂₈ glyceride and one or more of the one or more further excipients form a mixture, preferably an intimate mixture, in the pharmaceutical composition. Preferably the mixture or the intimate mixture is suitable for direct compression into tablets.

Optionally the pharmaceutical composition of the present invention comprises granules or particles comprising the ACE inhibitor, or the pharmaceutically acceptable salt or derivative thereof, wherein the granules or particles comprise a coating comprising the C₁₆-C₂₈ glyceride. The granules or particles may optionally further comprise one or more excipients.

Preferably the composition is a solid composition, more preferably it is a non-effervescent composition.

Optionally the pharmaceutical composition of the present invention may further comprise a β-blocker, a diuretic, a calcium-channel blocker, a vasodilator anti-hypertensive drug, or an angiotensin II receptor antagonist.

Typically, the pharmaceutical composition of the present invention is suitable for oral, parental, transdermal, airway, rectal, vaginal or topical administration. Preferably the composition is suitable for oral administration.

A composition suitable for oral administration may be in unit dosage form comprising 1-20 mg, preferably 1-10 mg, of the ACE inhibitor or the pharmaceutically acceptable salt or derivative thereof. A composition suitable for oral administration is typically provided in the form of tablets, capsules, caplets, troches, lozenges, dragées, powder, granules or particles. Optionally the tablets, capsules, caplets, troches, lozenges, dragées, powder, granules or particles not only contain the C₁₆-C₂₈ glyceride, but also comprise a coating comprising the C₁₆-C₂₈ glyceride. Preferably the composition is provided in the form of tablets. Preferably the tablets have a disintegration time of not more than 10 Minutes, more preferably of not more than 5 minutes, in water at 36-38° C. Preferably the tablets have a shelf-life of at least 18 months, preferably of at least 24 months, more preferably of at least 4 or 5 years.

Preferably the composition is for use as a medicament, typically for the treatment or prevention of a cardiovascular disease, a coronary heart disease, a cerebrovascular disease, a peripheral vascular disease, arrhythmia, hypertension, cardiac failure, cardiovascular death, myocardial infarction, stroke or angina.

A further embodiment of the present invention provides a method of treating or preventing a cardiovascular disease, a coronary heart disease, a cerebrovascular disease, a peripheral vascular disease, arrhythmia, hypertension, cardiac failure, cardiovascular death, myocardial infarction, stroke or angina, comprising administering an effective amount of a pharmaceutical composition of the present invention to a patient in need thereof.

A further embodiment of the present invention provides a use of a pharmaceutical composition of the present invention in the manufacture of a medicament for the treatment or prevention of a cardiovascular disease, a coronary heart disease, a cerebrovascular disease, a peripheral vascular disease, arrhythmia, hypertension, cardiac failure, cardiovascular death, myocardial infarction, stroke or angina.

A further embodiment of the present invention provides a method of preparing a pharmaceutical composition of the present invention, comprising the step of blending the ACE inhibitor, or the pharmaceutically acceptable salt or derivative thereof, with the C₁₆-C₂₈ glyceride and optionally one or more further excipients. Preferably the method comprises the steps of blending the ACE inhibitor, or the pharmaceutically acceptable salt or derivative thereof, with the C₁₆-C₂₈ glyceride to form a pre-mix, and then blending the pre-mix with one or more further excipients. Preferably the ACE inhibitor, or the pharmaceutically acceptable salt or derivative thereof, the C₁₆-C₂₈ glyceride and optionally one or more further excipients are blended to form an intimate mixture. Preferably the method further comprises the step of compressing the blend of the ACE inhibitor, or the pharmaceutically acceptable salt or derivative thereof, and the excipient(s) into tablets by direct compression. Optionally the tablets, comprising a C₁₆-C₂₈ glyceride, may also be provided with a coating comprising a C₁₆-C₂₈ glyceride.

Alternatively, a method of preparing a pharmaceutical composition of the present invention may comprise the steps of preparing granules or particles comprising the ACE inhibitor, or the pharmaceutically acceptable salt or derivative thereof, and optionally one or more excipients, and providing the granules or particles with a coating comprising the C₁₆-C₂₈ glyceride.

The composition may be prepared by the methods of the present invention in batches of 5-150 kg, preferably in batches of 5-100 kg.

A further embodiment of the present invention provides a method of providing a stable pharmaceutical composition comprising an ACE inhibitor, or a pharmaceutically acceptable salt or derivative thereof, the method comprising incorporating a C₁₆-C₂₈ glyceride into the composition. Preferably the method of providing a stable pharmaceutical composition comprises incorporating the C₁₆-C₂₈ glyceride into the composition in a mixture, preferably an intimate mixture, with the ACE inhibitor or the pharmaceutically acceptable salt or derivative thereof. Preferably the pharmaceutical composition is stabilised to minimize the degradation of the ACE inhibitor or the pharmaceutically acceptable salt or derivative thereof. Preferably the ACE inhibitor, or the pharmaceutically acceptable salt or derivative thereof, is susceptible to heat and/or mechanical stress-induced degradation. More preferably the ACE inhibitor, or the pharmaceutically acceptable salt or derivative thereof, is ramipril, trandolapril, quinapril, or a pharmaceutically acceptable salt or derivative thereof. In the most preferred embodiment of the present invention, the ACE inhibitor, or the pharmaceutically acceptable salt or derivative thereof, is ramipril or a pharmaceutically acceptable salt or derivative thereof. Preferably the pharmaceutical composition comprises 5-30% by weight C₁₆-C₂₈ glyceride, more preferably 5-20% by weight, even more preferably 10-15% by weight of the total composition. Preferably the glyceride is a C₁₈-C₂₆ glyceride, more preferably a C₂₀-C₂₄ glyceride, even more preferably a C₂₂ glyceride. Preferably the glyceride comprises at least 50% diglyceride, more preferably at least 60% diglyceride, even more preferably at least 70% diglyceride. In the most preferred embodiment of the present invention, the glyceride is glycerol dibehenate.

The present invention further provides a use of a C₁₆-C₂₈ glyceride to provide a stable pharmaceutical composition comprising an ACE inhibitor or a pharmaceutically acceptable salt or derivative thereof. Preferably the pharmaceutical composition is stabilised to minimize the degradation of the ACE inhibitor or the pharmaceutically acceptable salt or derivative thereof. Preferably the ACE inhibitor, or the pharmaceutically acceptable salt or derivative thereof, is susceptible to heat and/or mechanical stress-induced degradation. More preferably the ACE inhibitor, or the pharmaceutically acceptable salt or derivative thereof, is ramipril, trandolapril, quinapril, or a pharmaceutically acceptable salt or derivative thereof. In the most preferred embodiment of the present invention, the ACE inhibitor, or the pharmaceutically acceptable salt or derivative thereof, is ramipril or a pharmaceutically acceptable salt or derivative thereof. Preferably the pharmaceutical composition comprises 5-30% by weight C₁₆-C₂₈ glyceride, more preferably 5-20% by weight, even more preferably 10-15% by weight of the total composition. Preferably the glyceride is a C₁₈-C₂₆ glyceride, more preferably a C₂₀-C₂₄ glyceride, even more preferably a C₂₂ glyceride. Preferably the glyceride comprises at least 50% diglyceride, more preferably at least 60% diglyceride, even more preferably at least 70% diglyceride. In the most preferred embodiment of the present invention, the glyceride is glycerol dibehenate.

### BRIEF DESCRIPTION OF THE DRAWINGS

The present invention will now be described by way of example with reference to the accompanying drawings in which:

FIG. 1 schematically depicts some degradation pathways of ramipril, in particular to degradation products D, E, F, K and L. A list of degradation products and impurities A to N of ramipril is provided in European Pharmacopoeia, 2002, 4^{th} edition.

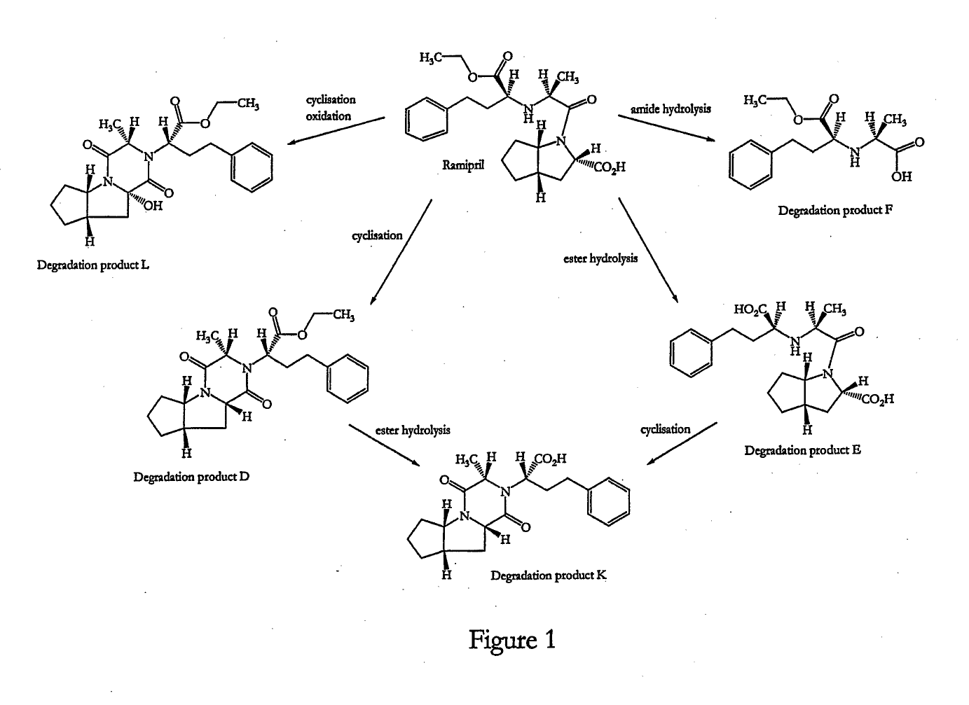

FIG. 2 is a graph showing the increase in total impurities (%) in tablets of formulations 1-4 and 19 when stored at 40° C. and 75% relative humidity.

### DETAILED DESCRIPTION OF THE INVENTION

It has now surprisingly been found that the presence of a C₁₆-C₂₈ glyceride, such as glycerol dibehenate, reduces or slows the degradation of certain ACE inhibitors in pharmaceutical compositions.

Glycerol dibehenate, also called glyceryl dibehenate, glyceryl behenate and 2,3-dihydroxypropyl docosanoate, is sold under the trade name Comptitol®. PharmEuropa (section 9, 2001) describes glyceryl dibehenate as a mixture of diacylglycerols, mainly dibehenoylglycerol, together with variable quantities of mono- and triacylglycerols. The US Pharmacopeia 24/National Formulary 19 describes glyceryl behenate as a mixture of glycerides of fatty acids, mainly behenic acid and specifies that the content of 1-monoglycerides should be between 12.0-18.0%.

Glycerol dibehenate is used in cosmetics, foods and oral pharmaceutical formulations and is generally regarded as a relatively non-irritant and non-toxic material. It is GRAS listed and included in the FDA's Inactive Ingredients Guide.

In pharmaceutical formulations, glycerol dibehenate is mainly used as a tablet or capsule lubricant, tablet binder or coating agent. It has also been investigated for use in the preparation of sustained release tablets.

Without wishing to be bound by theory, it is believed that C₁₆-C₂₈ glycerides, such as glycerol dibehenate, may reduce or slow the degradation of certain ACE inhibitors and their salts and derivatives in pharmaceutical compositions as follows.

Certain ACE inhibitors and their salts and derivatives are unstable and susceptible to degradation, especially in the presence of heat and mechanical stress such as, for example, due to pressure and heat exerted during compression of a powder blend into tablets. C₁₆-C₂₈ glycerides, such as glycerol dibehenate, are plastically deformable compounds. Therefore, when pharmaceutical compositions comprising such an ACE inhibitor, or a pharmaceutically acceptable salt or derivative thereof, and a C₁-C₂₈ glyceride, are compressed into tablets, the C₁₆-C₂₈ glyceride reduces the compression heat and mechanical stress due to its plastic deformability. In other words, the C₁₆-C₂₈ glyceride acts as a cushioning agent to protect the unstable ACE inhibitor, or its salt or derivative, from heat and mechanical stress.

Compatibility Studies

### EXAMPLE 1

Commercially available formulations of ramipril are currently sold by Aventis, Hoechst and Astra under trade names such as Tritace®, Acovil®, Delix® or Ramace®. These commercially available formulations contain ramipril as active ingredient as well as hydroxypropylmethylcellulose, pregelatinised starch, microcrystalline cellulose, sodium steatyl fumarate, yellow ferric oxide and red ferric oxide as inactive ingredients.

Ramipril tablets of formulations 1 to 4 were prepared with a composition similar to these commercially available ramipril formulations. Ramipril tablets 1 to 4 all comprise ramipril as well as pregelatinised starch, microcrystalline cellulose, sodium stearyl fumarate and yellow ferric oxide as inactive ingredients, as summarised in Table 1. Ramipril tablets 1 to 4 were prepared by mixing ramipril and the excipients intimately and then compressing the drug/excipient blend into tablets.

TABLE 1

| Ingredients (mg/tablet) | Tablet 1 | Tablet 2 | Tablet 3 | Tablet 4 |
|---|---|---|---|---|
| Ramipril | 2.5 | 5.0 | 2.5 | 5.0 |
| Pregelatinised starch | 30.9 | 61.8 | 45.9 | 91.8 |
| Microcrystalline cellulose | 65.0 | 130.0 | 50.0 | 100.0 |
| Sodium stearyl fumarate | 1.0 | 2.0 | 1.0 | 2.0 |
| Yellow ferric oxide | 0.6 | 1.2 | 0.6 | 1.2 |
| Total weight | 100.0 | 200.0 | 100.0 | 200.0 |

The stability of ramipril in the tablets of formulations 1 to 4 stored in PVdC-coated PVC/aluminium blister packs at 25° C. and 60% relative humidity, at 30° C. and 60% relative humidity, and at 40° C. and 75% relative humidity was studied following the procedures described in the ICH Guidelines (International Conference on Harmonisation of Technical Standards Guidelines).

The results of the stability studies of ramipril tablets 1 to 4 are presented in Table 2. As can bee seen, although immediately after compression of the tablets 1 to 4 there is no significant increase in total impurities, after 6 weeks storage at 40° C. and 75% relative humidity, the total impurities in all of tablets 1 to 4 have increased to more than 2.5%. This level of degradation is high and stabilisation of the drug is desirable.

TABLE 2

| | | | | Total | |
|---|---|---|---|---|---|
| | | | Total | Un- | |
| | | | Known | known | Total |
| | | | Im- | Im- | Im- |
| | Storage | | purities | purities | purities |
| Tablet | conditions | Time | (%) | (%) | (%) |
| Tablet 1 | Initial | Initial | 0.64 | 0.00 | 0.64 |
| | 25° C./60% RH | After 2 weeks | 0.84 | 0.00 | 0.84 |
| | 40° C./75% RH | | 2.21 | 0.00 | 2.21 |
| | 25° C./60% RH | After 4 weeks | 1.03 | 0.00 | 1.03 |
| | 40° C./75% RH | | 3.00 | 0.07 | 3.07 |
| | 25° C./60% RH | After 6 weeks | 1.36 | 0.00 | 1.36 |
| | 30° C./60% RH | | 1.67 | 0.00 | 1.67 |
| | 40° C./75% RH | | 3.36 | 0.18 | 3.54 |
| Tablet 2 | Initial | Initial | 0.54 | 0.00 | 0.54 |
| | 25° C./60% RH | After 2 weeks | 1.06 | 0.00 | 1.06 |
| | 40° C./75% RH | | 2.04 | 0.00 | 2.04 |
| | 25° C./60% RH | After 4 weeks | 1.05 | 0.06 | 1.11 |
| | 40° C./75% RH | | 3.43 | 0.06 | 3.49 |
| | 25° C./60% RH | After 6 weeks | 1.53 | 0.00 | 1.53 |
| | 30° C./60% RH | | 1.78 | 0.00 | 1.78 |
| | 40° C./75% RH | | 3.27 | 0.25 | 3.52 |
| Tablet 3 | Initial | Initial | 0.48 | 0.00 | 0.48 |
| | 25° C./60% RH | After 2 weeks | 0.48 | 0.00 | 0.48 |
| | 40° C./75% RH | | 0.96 | 0.00 | 0.96 |
| | 25° C./60% RH | After 4 weeks | 0.85 | 0.00 | 0.85 |
| | 30° C./60% RH | | 1.09 | 0.00 | 1.09 |
| | 40° C./75% RH | | 2.30 | 0.05 | 2.35 |
| | 25° C./60% RH | After 6 weeks | 0.85 | 0.00 | 0.85 |
| | 30° C./60% RH | | 1.08 | 0.00 | 1.08 |
| | 40° C./75% RH | | 2.58 | 0.06 | 2.65 |
| Tablet 4 | Initial | Initial | 0.56 | 0.00 | 0.56 |
| | 25° C./60% RH | After 2 weeks | 0.98 | 0.00 | 0.98 |
| | 40° C./75% RH | | 1.66 | 0.00 | 1.66 |
| | 25° C./60% RH | After 4 weeks | 0.85 | 0.00 | 0.85 |
| | 30° C./60% RH | | 1.07 | 0.06 | 1.12 |
| | 40° C./75% RH | | 2.25 | 0.05 | 1.12 |
| | 25° C./60% RH | After 6 weeks | 0.93 | 0.00 | 0.93 |
| | 30° C./60% RH | | 1.14 | 0.05 | 1.19 |
| | 40° C./75% RH | | 2.67 | 0.07 | 2.74 |

### EXAMPLE 2

Ramipril tablets of formulations 5 to 18, comprising ramipril and excipients as set out in Table 3, were prepared and the effect of heat and mechanical stress on drug stability in these tablets was studied in order to identify excipients that have a stabilising effect on ramipril. Unless otherwise indicated in Table 3, ramipril tablets 5 to 18 were prepared by mixing ramipril and the excipients intimately and then compressing the drug/excipient blend into tablets.

The stability of ramipril in tablets 5 to 18 stored in high-density polyethylene containers at 40° C. and 75% relative humidity was studied following the procedures described in the ICH Guidelines. The results of the stability studies of ramipril tablets 5 to 18 are presented in Table 4.

Based on the results presented in Table 4, it can be concluded that the addition of pH modulators like sodium bicarbonate, lysine monohydrate, magnesium carbonate etc. can help in controlling levels of impurity D, the major heat degradation product of ramipril. However, all of these pH modulators cause a significant increase in impurities E and F, hydrolytic degradation products of ramipril, which are known to occur in alkaline conditions.

Two formulations, tablets 6 and 14, showed a lesser amount of ramipril degradation compared to all other formulations. In both cases, heat degradation product D was found to be less than 1.5% after 8 weeks storage at 40° C. and 75% relative humidity, whereas it was almost 6% for formulations 7 and 16. A disadvantage of formulation 14 is that the amount of hydrolytic degradation products E and F increases to almost 1% after 8 weeks storage at 40° C. and 75% relative humidity, due to the alkaline pH of formulation of 14. Formulation 6, on the other hand, does not show any hydrolytic degradation products E or F, and the levels of heat degradation product D are also very low.

TABLE 3

| Ingredients (mg/tablet) | T 5 | T 6 | T 7 | T 8^{b} | T 9^{b} | T 10^{b} | T 11 | T 12^{c} | T 13^{c} | T 14^{b,d} | T 15^{b} | T 16^{e} | T 17 | T 18 |
|---|---|---|---|---|---|---|---|---|---|---|---|---|---|---|
| Ramipril | 5.0 | 5.0 | 5.0 | 5.0 | 5.0 | 5.0 | 5.0 | 5.0 | 5.0 | 5.0 | 5.0 | 5.0 | 5.0 | 5.0 |
| Anhydrous lactose | 179.0 | 164.0 | 189.0 | 164.0 | — | — | — | — | — | 184.0 | 184.0 | 149.0 | 139.0 | — |
| Sodium starch glycolate | 4.0 | 4.0 | 4.0 | 4.0 | — | — | — | — | — | 4.0 | 4.0 | — | 4.0 | — |
| Sodium stearyl fumarate | 2.0 | 2.0 | 2.0 | 2.0 | 2.0 | 2.0 | 2.0 | 2.0 | 2.0 | 2.0 | 2.0 | 2.0 | 2.0 | 2.0 |
| Stearic acid | 10.0 | — | — | — | — | — | — | — | — | — | — | — | — | — |
| Glycerol dibehenate | — | 25.0 | — | — | — | — | — | — | — | — | — | — | — | — |
| Lysine monohydrate | — | — | — | 25.0 | — | — | — | — | — | — | — | — | — | — |
| Sodium bicarbonate | — | — | — | — | 5.0 | 50.0 | — | — | — | — | — | — | — | — |
| Tricalcium phosphate | — | — | — | — | 184.0 | 139.0 | 189.0 | — | — | — | — | — | — | — |
| Crospovidone | — | — | — | — | 4.0 | 4.0 | 4.0 | — | — | — | — | — | — | — |
| Alcoholic solution of polyvinyl | — | — | — | — | — | — | — | 10.0 | — | — | — | — | — | — |
| pyrrolidone | | | | | | | | | | | | | | |
| Alcoholic solution of | — | — | — | — | — | — | — | — | 10.0 | — | — | — | — | — |
| Carbopol ® | | | | | | | | | | | | | | |
| Microcrystalline cellulose | — | — | — | — | — | — | — | 115.0 | 115.0 | — | — | 44.0 | — | 123.94 |
| Pregelled starch | — | — | — | — | — | — | — | 68.0 | 68.0 | — | — | — | — | 68.3 |
| TRIS buffer | — | — | — | — | — | — | — | — | — | 5.0 | 5.0 | — | — | — |
| Magnesium carbonate | — | — | — | — | — | — | — | — | — | — | — | — | 50.0 | — |
| Hydroxypropylmethylcellulose | — | — | — | — | — | — | — | — | — | — | — | — | — | 0.76 |
| Total weight | 200.0 | 200.0 | 200.0 | 200.0 | 200.0 | 200.0 | 200.0 | 200.0 | 200.0 | 200.0 | 200.0 | 200.0 | 200.0 | 200.0 |

a - Abbreviations used: T = tablet.
^{b}The pH of formulations 8, 9, 10, 14 and 15 was 9.4, 6.2, 8.3, 8.4 and 8.4 respectively.
^{c}The drug was granulated with the alcoholic solution, the granules were dried, intimately mixed with the other excipients and compressed into tablets 12 and 13.
^{d}Formulation 14 was prepared by wet granulation.
^{e}Low moisture excipients were used to control the moisture content of formulation 16.

TABLE 4

| | | Initial | | | After 2 weeks | | | After 4 weeks | | | After 6 weeks | | | After 8 weeks | | |
|---|---|---|---|---|---|---|---|---|---|---|---|---|---|---|---|---|
| | | | Imp. | Total | | Imp. | Total | | Imp. | Total | | Imp. | Total | | Imp. | Total |
| Tab. | Composition | Imp. D | E + F | Imp. | Imp. D | E + F | Imp. | Imp. D | E + F | Imp. | Imp. D | E + F | Imp. | Imp. D | E + F | Imp. |
| 5 | ramipril, stearic acid | 0.86 | 0.03 | 1.10 | 0.83 | 0.03 | 1.85 | 36.23 | 0.14 | 38.38 | — | — | — | — | — | — |
| | (1:2), AL, SSG, SSF | | | | | | | | | | | | | | | |
| 6 | ramipril, glycerol | 0.12 | 0.02 | 0.26 | 0.25 | 0.01 | 0.46 | 0.39 | 0.02 | 0.55 | 0.70 | 0.02 | 0.98 | 1.25 | 0.00 | 1.76 |
| | dibehenate (1:5), | | | | | | | | | | | | | | | |
| | AL, SSG, SSF | | | | | | | | | | | | | | | |
| 7 | ramipril, AL, SSG, | 0.19 | 0.02 | 0.35 | 1.77 | 0.09 | 2.09 | 3.72 | 0.15 | 4.15 | 4.60 | 0.19 | 5.35 | 5.98 | 0.22 | 6.82 |
| | SSF (NC) | | | | | | | | | | | | | | | |
| 8 | ramipril, lysine | 0.08 | 0.10 | 0.31 | — | — | — | — | — | — | — | — | — | — | — | — |
| | monohydrate (1:5), | | | | | | | | | | | | | | | |
| | AL, SSG, SSF | | | | | | | | | | | | | | | |
| 9 | ramipril, sodium | 0.09 | 0.28 | 0.61 | 0.18 | 4.13 | 4.54 | — | — | — | — | — | — | — | — | — |
| | bicarbonate (1:1), | | | | | | | | | | | | | | | |
| | TP, CP, SSF | | | | | | | | | | | | | | | |
| 10 | ramipril, sodium | 0.07 | 0.33 | 0.64 | 0.11 | 2.17 | 2.43 | — | — | — | — | — | — | — | — | — |
| | bicarbonate (1:10), | | | | | | | | | | | | | | | |
| | TP, CP, SSF | | | | | | | | | | | | | | | |
| 11 | ramipril, TP, CP, | 0.12 | 0.24 | 0.64 | 0.20 | 4.51 | 5.05 | — | — | — | — | — | — | — | — | — |
| | SSF (NC) | | | | | | | | | | | | | | | |
| 12 | ramipril, PVP (1:2), | 0.62 | 0.05 | 0.79 | 15.36 | 0.53 | 16.38 | — | — | — | — | — | — | — | — | — |
| | MCC, PS, SSF | | | | | | | | | | | | | | | |
| 13 | ramipril, Carbopol ® | 0.09 | 0.05 | 0.23 | 0.43 | 0.60 | 1.14 | 0.95 | 1.69 | 2.88 | 1.22 | 2.39 | 3.95 | — | — | — |
| | (1:2), MCC, PS, SSF | | | | | | | | | | | | | | | |
| 14 | ramipril, TRIS buffer | 0.00 | 0.03 | 0.08 | 0.07 | 0.11 | 0.25 | 0.29 | 0.41 | 1.00 | 0.49 | 0.69 | 1.53 | 0.85 | 0.98 | 2.47 |
| | (1:1), AL, SSG, SSF | | | | | | | | | | | | | | | |
| | (wet granulation) | | | | | | | | | | | | | | | |
| 15 | ramipril, TRIS buffer | 0.09 | 0.02 | 0.23 | 0.11 | 0.16 | 0.37 | 0.65 | 0.71 | 1.94 | 1.11 | 1.37 | 3.39 | — | — | — |
| | (1:1), AL, SSG, SSF | | | | | | | | | | | | | | | |
| | (dry mix) | | | | | | | | | | | | | | | |
| 16 | ramipril, AL, MCC, | 0.10 | 0.02 | 0.26 | 1.57 | 0.08 | 1.73 | 3.38 | 0.15 | 3.74 | 4.95 | 0.21 | 5.50 | 5.58 | 0.24 | 6.20 |
| | SSF (LM) | | | | | | | | | | | | | | | |
| 17 | ramipril, magnesium | 0.05 | 0.09 | 0.27 | 0.11 | 0.73 | 0.96 | 0.18 | 2.58 | 2.99 | 0.21 | 3.07 | 3.59 | — | — | — |
| | carbonate (1:10), AL, | | | | | | | | | | | | | | | |
| | SSG, SSF | | | | | | | | | | | | | | | |
| 18 | ramipril, HPMC | 0.11 | 0.02 | 0.30 | 1.41 | 0.07 | 1.59 | 2.67 | 0.11 | 2.97 | 2.72 | 0.09 | 3.21 | — | — | — |
| | (1:0.76), MCC, PS, | | | | | | | | | | | | | | | |
| | SSF | | | | | | | | | | | | | | | |

Abbreviations used:
AL = anhydrous lactose;
SSG = sodium starch glycolate;
SSF = sodium stearyl fumarate;
TP = tricalcium phosphate;
CP = crospovidone;
PVP = polyvinylpyrrolidone;
MCC = microcrystalline cellulose;
PS = pregelled starch;
HPMC = hydroxypropylmethylcellulose;
NC = negative control;
LM = low moisture;
Imp. = impurity;
Tab. = tablet.

Indeed formulation 6, comprising glycerol dibehenate, was found to be mote stable than any of the other formulations, showing no hydrolytic degradation to impurities E and F, and only minimal heat degradation to impurity D. Since the only difference between formulation 6 and less stable formulation 7 is the presence of glycerol dibehenate in formulation 6, it can be concluded that glycerol dibehenate has a stabilising effect on ramipril.

### EXAMPLE 3

To confirm the stabilising effect of glycerol dibehenate, ramipril tablets of formulations 19 to 23, comprising ramipril, glycerol dibehenate and other excipients as summarised in Table 5, were prepared and the effect of heat and mechanical stress on drug stability in these tablets was studied. Ramipril tablets 19 to 23 were prepared by pre-mixing ramipril and glycerol dibehenate intimately, followed by mixing the ramipril/glycerol dibehenate pre-mix with the remaining excipients intimately, and then compressing the drug/excipient blend into tablets.

TABLE 5

| | | Tablet | Tablet | Tablet | Tablet |
|---|---|---|---|---|---|
| Ingredients (mg/tablet) | Tablet 19 | 20 | 21 | 22 | 23 |
| Ramipril | 5.0 | 10.0 | 5.0 | 2.5 | 1.25 |
| Compritol 888 ATO ® | 25.0 | 25.0 | 25.0 | 12.5 | 6.25 |
| Pharmatose DCL 21 ® | 144.0 | 139.0 | 143.8 | 71.6 | 36.0 |
| Primojel ® | 24.0 | 24.0 | 24.0 | 12.0 | 6.0 |
| PRUV ® | 2.0 | 2.0 | 2.0 | 1.0 | 0.5 |
| Red ferric oxide | — | — | 0.2 | — | — |
| Yellow ferric oxide | — | — | — | 0.4 | — |
| Total weight | 200.0 | 200.0 | 200.0 | 100.0 | 50.0 |

Compritol 888 ATO ® is glycerol dibehenate;
Pharmatose DCL 21 ® is anhydrous lactose;
Primojel ® is sodium starch glycolate;
PRUV ® is sodium stearyl fumarate;
and red ferric oxide and yellow ferric oxide are colouring agents.

The stability of ramipril in tablet 19 stored in high-density polyethylene containers at 25° C. and 60%/relative humidity, at 30° C. and 60% relative humidity, and at 40° C. and 75% relative humidity was studied following the procedures described in the ICH Guidelines. The results of the stability studies of ramipril tablet 19 are presented in Table 6.

TABLE 6

| | | | | Total |
|---|---|---|---|---|
| Storage | | Total Known | Total Unknown | Impurities |
| conditions | Time | Impurities (%) | Impurities (%) | (%) |
| Initial | Initial | 0.30 | 0.00 | 0.30 |
| 25° C./60% RH | After 2 | 0.34 | 0.00 | 0.34 |
| 40° C./75% RH | weeks | 0.53 | 0.00 | 0.53 |
| 25° C./60% RH | After 4 | 0.39 | 0.00 | 0.39 |
| 40° C./75% RH | weeks | 0.60 | 0.06 | 0.66 |
| 25° C./60% RH | After 6 | 0.39 | 0.00 | 0.39 |
| 40° C./75% RH | weeks | 0.88 | 0.09 | 0.97 |
| 25° C./60% RH | After 8 | 0.47 | 0.00 | 0.47 |
| 30° C./60% RH | weeks | 0.52 | 0.00 | 0.52 |
| 40° C./75% RH | | 1.28 | 0.16 | 1.44 |
| 25° C./60% RH | After 12 | 0.48 | 0.00 | 0.48 |
| 30° C./60% RH | weeks | 0.55 | 0.00 | 0.55 |
| 40° C./75% RH | | 1.95 | 0.40 | 2.35 |

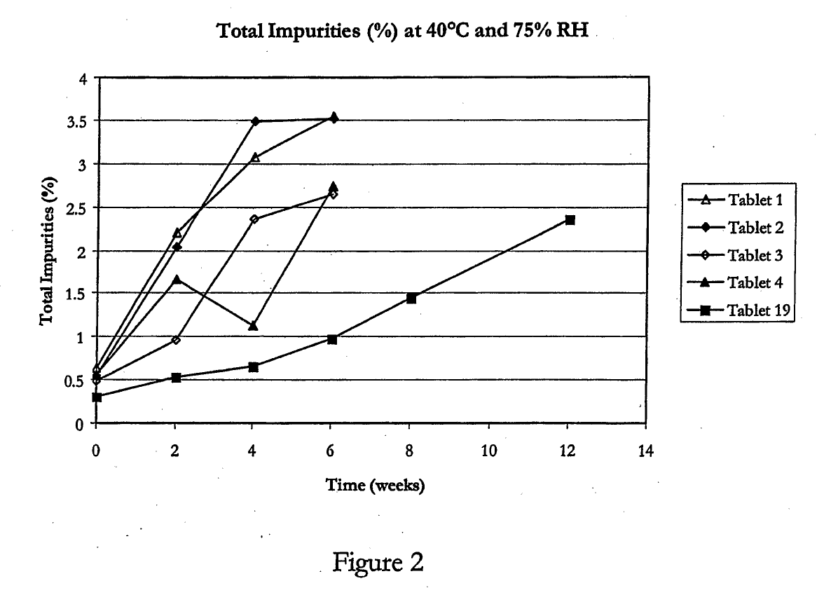

The stability of ramipril in tablets 1-4 and 19 is compared in FIG. 2, which shows the increase in total impurities (%) in the tablets when stored at 40° C. and 75% relative humidity. As can be seen, ramipril in tablet 19 is much more stable to degradation than ramipril in tablets 1-4, which have a composition similar to currently commercially available ramipril formulations.

The stability of ramipril in tablets 20 to 23 stored in high-density polyethylene containers at 40° C. and 75% relative humidity was studied following the procedures described in the ICH Guidelines. The percentage increase of heat degradation product D is summarised in Table 7.

TABLE 7

| | Impurity D (%) | | | |
|---|---|---|---|---|
| Time | Tablet 20 | Tablet 21 | Tablet 22 | Tablet 23 |
| Initial | 0.24 | 0.30 | 0.28 | 0.30 |
| After 2 weeks | 0.42 | 0.32 | 0.27 | 0.40 |
| After 4 weeks | 0.51 | 0.67 | 0.35 | 0.59 |
| After 6 weeks | 0.72 | 0.95 | 0.44 | 0.86 |
| After 8 weeks | 0.87 | 0.95 | 0.42 | 1.03 |
| After 12 weeks | 1.15 | 1.68 | 0.59 | 1.60 |

The results presented in Tables 6 and 7 confirm that glycerol dibehenate reduces the degradation of ramipril in pharmaceutical compositions.

It will be understood that the present invention has been described above by way of example only. The examples are not intended to limit the scope of the invention. Various modifications and embodiments can be made without departing from the scope and spirit of the invention, which is defined by the following claims only.

1. A pharmaceutical composition comprising an ACE inhibitor, or a pharmaceutically acceptable salt or derivative thereof, and a C₁₆-C₂₈ glyceride, wherein the ACE inhibitor, or the pharmaceutically acceptable salt or derivative thereof, is ramipril, trandolapril, quinapril, or a pharmaceutically acceptable salt or derivative thereof.

2. A pharmaceutical composition of claim 1, comprising 5-30% by weight C₁₆-C₂₈ glyceride.

3. A pharmaceutical composition of claim 1, comprising one or more further excipients which are compatible with the ACE inhibitor or the pharmaceutically acceptable salt or derivative thereof.

4. A pharmaceutical composition of claim 3, wherein the one or more further excipients are selected from hydroxypropylmethylcellulose, pregelatinised starch, microcrystalline cellulose, lactose, sodium starch glycolate, sodium stearyl fumarate, red ferric oxide and yellow ferric oxide.

5. A pharmaceutical composition of claim 1, comprising:
2-6% by weight ACE inhibitor,
10-15% by weight C₁₆-C₂₈ glyceride,
65-75% by weight lactose anhydrous,
10-15% by weight sodium starch glycolate,
0.5-2% by weight sodium stearyl fumarate,
0-0.4% by weight yellow ferric oxide, and
0-0.1% by weight red ferric oxide.

6. A pharmaceutical composition of claim 1, wherein the pharmaceutical composition is stable.

7. A pharmaceutical composition of claim 1, suitable for direct compression into tablets.

8. A pharmaceutical composition of claim 1, wherein the ACE inhibitor, or the pharmaceutically acceptable salt or derivative thereof, and the C₁₆-C₂₈ glyceride form a mixture.

9. A pharmaceutical composition of claim 1, wherein the ACE inhibitor, or the pharmaceutically acceptable salt or derivative thereof, the C₁₆-C₂₈ glyceride and one or more further excipients form a mixture.

10. A pharmaceutical composition of claim 8 or 9, wherein the mixture is an intimate mixture.

11. A pharmaceutical composition of claim 8 or 9, wherein the mixture is suitable for direct compression into tablets.

12. A pharmaceutical composition of claim 1, comprising granules or particles comprising the ACE inhibitor, or the pharmaceutically acceptable salt or derivative thereof, wherein the granules or particles comprise a coating comprising the C₁₆-C₂₈ glyceride.

13. A pharmaceutical composition of claim 1, further comprising a O-blocker, a diuretic, a calcium-channel blocker, a vasodilator anti-hypertensive drug, or an angiotensin II receptor antagonist.

14. A pharmaceutical composition of claim 1, wherein the composition is suitable for oral, parental, transdermal, airway, rectal, vaginal or topical administration.

15. A pharmaceutical composition of claim 1, wherein the composition is suitable for oral administration.

16. A pharmaceutical composition of claim 15, wherein the composition is in unit dosage form comprising 1-20 mg of the ACE inhibitor or the pharmaceutically acceptable salt or derivative thereof.

17. A pharmaceutical composition of claim 15, wherein the composition is provided in the form of a tablet, capsule, caplet, troche, lozenge, dragée, powder, granules or particles.

18. A pharmaceutical composition of claim 17, wherein the tablet, capsule, caplet, troche, lozenge, dragée, powder, granules or particles comprise a coating comprising the C₁₆-C₂₈ glyceride.

19. A pharmaceutical composition of claim 15, wherein the composition is provided in the form of a tablet.

20. A pharmaceutical composition of claim 19, wherein the tablet has a disintegration time of not more than 10 minutes.

21. A pharmaceutical composition of claim 19, wherein the tablet has a shelf-life of at least 5 years.

22. (canceled)

23. A pharmaceutical composition of claim 1, for use as a medicament.

24. A pharmaceutical composition of claim 23, for use as a medicament for the treatment or prevention of a cardiovascular disease, a coronary heart disease, a cerebrovascular disease, a peripheral vascular disease, arrhythmia, hypertension, cardiac failure, cardiovascular death, myocardial infarction, stroke or angina.

25. A method of treating or preventing a cardiovascular disease, a coronary heart disease, a cerebrovascular disease, a peripheral vascular disease, arrhythmia, hypertension, cardiac failure, cardiovascular death, myocardial infarction, stroke or angina, comprising administering an effective amount of a pharmaceutical composition of claim 1 to a patient in need thereof.

26. (canceled)

27. A method of preparing a pharmaceutical composition of a claim 1, comprising the step of blending the ACE inhibitor, or the pharmaceutically acceptable salt or derivative thereof, with the C₁₆-C₂₈ glyceride.

28. A method of claim 27, wherein the ACE inhibitor, or the pharmaceutically acceptable salt or derivative thereof, and the C₁₆-C₂₈ glyceride are blended to form an intimate mixture.

29. A method of claim 27, comprising the step of blending the ACE inhibitor, or the pharmaceutically acceptable salt or derivative thereof, with the C₁₆-C₂₈ glyceride and one or more further excipients.

30. A method of claim 29, comprising the steps of blending the ACE inhibitor, or the pharmaceutically acceptable salt or derivative thereof, with the C₁₆-C₂₈ glyceride to form a pre-mix, and blending the pre-mix with one or more further excipients.

31. A method of claim 29 or 30, wherein the ACE inhibitor, or the pharmaceutically acceptable salt or derivative thereof, the C₁₆-C₂₈ glyceride and one or more further excipients are blended to form an intimate mixture.

32. A method of claim 27, further comprising the step of compressing the blend of the ACE inhibitor, or the pharmaceutically acceptable salt or derivative thereof, and the excipient(s) into tablets by direct compression.

33. A method of claim 32, further comprising the step of providing the tablets with a coating comprising the C₁₆-C₂₈ glyceride.

34. A method of preparing a pharmaceutical composition of claim 1, comprising the steps of preparing granules or particles comprising the ACE inhibitor, or the pharmaceutically acceptable salt or derivative thereof, and optionally one or more excipients, and providing the granules or particles with a coating comprising the C₁₆-C₂₈ glyceride.

35. A method of claim 27 or 34, wherein the pharmaceutical composition is prepared in batches of 5-150 kg.

36. A method of providing a stable pharmaceutical composition comprising an ACE inhibitor or a pharmaceutically acceptable salt or derivative thereof, wherein the ACE inhibitor, or the pharmaceutically acceptable salt or derivative thereof, is ramipril, trandolapril, quinapril, or a pharmaceutically acceptable salt or derivative thereof the method comprising incorporating a C₁₆-C₂₈ glyceride into the composition.

37. A method of claim 36, comprising incorporating the C₁₆-C₂₈ glyceride into the composition in a mixture with the ACE inhibitor or the pharmaceutically acceptable salt or derivative thereof.

38. A method of claim 37, comprising incorporating the C₁₆-C₂₈ glyceride into the composition in an intimate mixture with the ACE inhibitor or the pharmaceutically acceptable salt or derivative thereof.

39. (canceled)

40. A method of claim 36, wherein the pharmaceutical composition is stabilised to minimize the degradation of the ACE inhibitor or the pharmaceutically acceptable salt or derivative thereof.

41. A pharmaceutical composition of claim 1, wherein the ACE inhibitor, or the pharmaceutically acceptable salt or derivative thereof, is susceptible to heat and/or mechanical stress-induced degradation.

42. (canceled)

43. A pharmaceutical composition of claim 1, wherein the ACE inhibitor, or the pharmaceutically acceptable salt or derivative thereof, is ramipril or a pharmaceutically acceptable salt or derivative thereof.

44. A pharmaceutical composition of claim 1, wherein the glyceride comprises one, two or three C₁₆-C₂₈ acyl moieties, wherein each C₁₆-C₂₈ acyl moiety is independently of the formula —CO—R, wherein R is a saturated or unsaturated hydrocarbon, which contains from 16 to 28 carbon atoms, and which is straight-chained or branched.

45. A pharmaceutical composition of claim 44, wherein R is a saturated hydrocarbon and/or wherein R is a straight-chained hydrocarbon.

46. A pharmaceutical composition of claim 1, wherein the glyceride is a C₁₈-C₂₆ glyceride.

47. A pharmaceutical composition of claim 46, wherein the glyceride is a C₂₀-C₂₄ glyceride.

48. A pharmaceutical composition of claim 1, wherein the glyceride comprises at least 50% diglyceride.

49. A pharmaceutical composition of claim 1, wherein the glyceride is glycerol dibehenate.
